(19) United States
(12) Patent Application Publication
(10) Pub. No.: US 2024/0201176 A1
(43) Pub. Date: Jun. 20, 2024

(54) METHOD AND DEVICE FOR SYNCHRONOUS EXTRACTION AND ANALYSIS OF MULTIPLE TRACE EMERGING ORGANIC CONTAMINANTS IN HAIRS, AND APPLICATION

(71) Applicant: SOUTH CHINA INST OF ENVIRONMENTAL SCIENCES MEE ECOLOGY AND ENVIRONMENT EMERGENCY INST OF ECOLOGY, Guangzhou (CN)

(72) Inventors: Jing ZHENG, Guangzhou (CN); Rongfa XU, Guangzhou (CN); Bin TANG, Guangzhou (CN); Xi ZHUANG, Guangzhou (CN); Xiao YAN, Guangzhou (CN); Weikeng LUO, Guangzhou (CN); Yunjiang YU, Guangzhou (CN)

(73) Assignee: SOUTH CHINA INST OF ENVIRONMENTAL SCIENCES MEE ECOLOGY AND ENVIRONMENT EMERGENCY INST OF ECOLOGY, Guangzhou (CN)

(21) Appl. No.: 18/334,365

(22) Filed: Jun. 13, 2023

(30) Foreign Application Priority Data
Dec. 15, 2022 (CN) 202211612399.X

(51) Int. Cl. G01N 33/53; G01N 1/34

(52) U.S. Cl. CPC G01N 33/5308; G01N 1/34

(57) ABSTRACT

A method for synchronous extraction and analysis of multiple trace emerging organic contaminants in hairs, and application comprises the following steps: S1, pretreatment; S2, mixing and extraction; S3, nitrogen blowing concentration and purification; and S4, low-temperature separation. A device for synchronous extraction and analysis of multiple trace emerging organic contaminants in hairs, and application comprises comprises a treatment barrel, a pedestal and a drive assembly. This method is applied to a study on long-term exposure of multiple organic contaminants in a human body. According to the present disclosure, an extraction solution extracting system is established, 5 emerging organic contaminants in hairs can be purified and analyzed by establishing a dispersion solid-phase extraction filler consisting of Na₂SO₄ powders and C18 powders.

### REFERENCE TO RELATED APPLICATIONS

The present application claims the priority of Chinese patent application No. 202211612399.X, filed on 2022 Dec. 15, the entire disclose of which is incorporated herein by reference.

### TECHNICAL FIELD OF THE INVENTION

The present disclosure relates to the technical field of biological monitoring of a human body, particularly to a method and device for synchronous extraction and analysis of multiple trace emerging organic contaminants in hairs, and application.

### BACKGROUND OF THE INVENTION

Emerging organic contaminants (EOCs) refer to organic contaminants that have emerged in environments or concerned in recent years, which may pose risks to ecosystems and human health, and are under insufficient management measures or have not yet been managed. The study suggests that these EOCs can cause endocrine disorders in a human body, and have neurotoxicity, reproductive toxicity, endocrine toxicity and carcinogenicity, etc.

Although there have been multiple literature reports on establishment of detection methods for EOCs in hairs, there are lacks of studies that can simultaneously extract and analyze multiple EOCs in hairs. Most of the existing literatures use nitric acid digestion to detect contaminants in hairs, such as PBDEs, OPFRs and PAEs, while alkaline digestion is used for PFASs. The two digestion methods have drawbacks such as time-consuming, solvent consuming, and lack of safety. In addition, studies have shown that the release of some endogenous substances while acid/alkali digestion destroys the structure of hairs may affect the detection of target contaminants such as PFASs in hairs, which has certain limitations; in contrast, hairs are extracted with an organic solvent after being ground, which can more accurately reflect the pollution level of PFASs in hairs. However, at present, there are lacks of relevant reports on whether this method is also applicable to other types of EOCs.

### SUMMARY OF THE INVENTION

For the above existing problems, the present disclosure provides a method and device for synchronous extraction and analysis of multiple trace emerging organic contaminants in hairs, and application.

The technical solution of the present disclosure is as follows:

Provided is a method for synchronous extraction and analysis of multiple trace emerging organic contaminants in hairs, comprising the following steps:
S1, pretreatment: adding a hair sample to be treated together with ultrapure water into a treatment barrel in a weight ratio of 1:20-40, cutting the hair sample through a cutting quartz knife in the treatment barrel while washing for 0.5-1 h, subsequently discharging ultrapure water and adding ultrapure whose weight is 20-40 times of the weight of the hair sample again, cutting the hair sample through the cutting quartz knife in the treatment barrel while washing, until the length of the hair sample is 0.5-1 cm, subsequently discharging the ultrapure water, and then grinding the hair sample for 5-10 min through a grinding plate in the treatment barrel to obtain hair powders;
S2, mixing and extraction: drying the hair powders obtained in step S1, placing 0.1 g of dried hair powders into a centrifuge tube, adding 1 mL of ethyl acetate and 1 mL of internal standard indicator into the centrifuge tube, standing for 12 h, and then adding 4 mL of extract solution into the centrifuge tube, wherein the extract solution is prepared from n-hexane, acetone, acetonitrile and ethyl acetate in a volume ratio of 1:1:1:1, and then starting to perform centrifugal extraction, wherein the steps for centrifugal extraction are as follows: vortex treatment is carried out for 4 min at the rotation speed of 2000 r·min⁻¹, then ultrasonic treatment is carried out for 20 min at the temperature of 20° C., and finally centrifugal treatment is carried out for 15 min at the rotation speed of 4000 r·min⁻¹, centrifugal extraction is repeated 3 times, and then the obtained supernatant is merged as a supernatant raw solution;
S3, nitrogen blowing concentration and purification: performing nitrogen blowing concentration on the supernatant raw solution obtained in step S2 to 1 mL, subsequently adding 20 mg of Na₂SO₄ powders and 100 mg of C18 powders serving as purifying fillers, then performing vortex treatment for 7 min at the rotation speed of 1200 r·min⁻¹, then performing centrifugal treatment for 15 min at the rotation speed of 3500 r·min⁻¹ to obtain a concentrated solution, and then transferring the concentrated solution to a glass tube; and
S4, low-temperature separation: blowing the concentrated solution obtained in step S3 with nitrogen for 1-2 h, then adding 200 μL of methanol solution with a mass fraction of >99.8% for redissolving, standing for 4-6 h at the temperature of −20° C., freezing and removing a precipitate to obtain the hair raw solution after low-temperature separation and performing Liquid chromatography-tandem mass spectrometry/mass spectrometry (LC-MS/MS) and Gas chromatography-tandem mass spectrometry/mass spectrometry (GC-MS/MS) detection on the hair raw solution.

As an aspect of the present disclosure, a method for preparing the internal standard indicator in the step S2 is as follows: mixing 0.1 mL of 50 μg/mL first internal standard raw solution, 0.1 mL of 50 μg/mL second internal standard raw solution, 0.05 mL of 100 μg/mL third internal standard raw solution, 0.05 mL of 100 μg/mL fourth internal standard raw solution and 0.05 mL of 100 μg/mL fifth internal standard raw solution and then adding 0.65 mL of methanol solution to obtain 1 mL of 5 μg/mL mixed internal standard, wherein the first internal standard raw solution is formed by mixing MPFOS and MPFOA in a volume ratio of 1:1, the second internal raw solution is formed by mixing dis-TCIPP, d₁₅-TDCIPP, d₁₅-TPhP and d₁₂-TCEP in a volume ratio of 1:1:1:1, the third internal standard raw solution is formed by mixing d₄-DEHP with d₄-DNBP in a volume ratio of 1:1, the fourth internal standard raw solution is formed by mixing ¹³C₄-mmBP, ¹³C₄-OH-mEHP, ¹³C-mEP, ¹³C-mBzP, d₄-mmOP, 50xo-MEHP-¹³C and MEHP-¹³C₄ in a volume ratio of 1:1:1:1:1:1:1, and the fifth internal standard raw solution is formed by mixing BDE118, BDE128 and ¹³C₁₂-BDE209 in a volume ratio of 1:1:1; and then diluting the mixed internal standard with a methanol solution to obtain an 100 ng/mL internal standard indicator.

Note: the used internal standard indicator is stable and easily available, and conforms to the method of the present disclosure.

The present disclosure further provides a device for use in the above method for synchronous extraction and analysis of multiple trace emerging organic contaminants in hairs, comprising a treatment barrel for washing, cutting and grinding a hair sample, a pedestal for supporting the treatment barrel, and a drive assembly located above the treatment barrel;
wherein, the middle inside the treatment barrel is provided with a rotating outer cylinder, the inside of the rotating outer cylinder is slidaly sleeved with a rotating inner cylinder that synchronously rotates with the rotating outer cylinder, the external circumference of the rotating outer cylinder is provided with a plurality of groups of hair cutting assemblies at equal intervals, the hair cutting assembly includes two auxiliary quartz knives and two cutting quartz knives, the auxiliary quartz knives and the cutting quartz knives are alternately arranged, the auxiliary quartz knives are arranged at the bottom and middle of the outer wall of the rotating outer cylinder, the upper and lower sides of the auxiliary quartz knives arranged in the middle of the outer wall of the rotating outer cylinder are both provided with sliding grooves, a connection block is arranged at the position of the sliding groove corresponding to the outer wall of the rotating inner cylinder, the connection block up and down slides along the sliding groove, the outer end of the connection block is fixedly connected with the cutting quartz knives, the auxiliary quartz knives and the cutting quartz knives both extend to the inner wall close to the treatment barrel, the auxiliary quartz knives and the cutting quartz knives on the same group of cutting assemblies are seamlessly engaged and aligned with each other through the blades, the blades provided on the cutting quartz knives are alternately engaged and aligned with the blades provided correspondingly on the auxiliary quartz knives when sliding up and down, the bottom of the treatment barrel is provided with a grinding plate, and the two sides of the bottoms of the auxiliary quartz knives located at the bottom of the outer wall of the rotating outer cylinder are provided with grinding portions that are matched with the grinding plate to grind the hair samples;
the top of the treatment barrel is provided with a top cover, the top of the rotating inner cylinder penetrates through the top cover and then is connected with the output end of a rotating motor of the drive assembly, the output end of the rotating motor is provided with a sleeve that is in up and down sliding connection with the rotating inner cylinder, the outer wall of the rotating inner cylinder located above the top cover is provided with a cylindrical telescopic gear lever, the telescopic gear lever includes an inner rod and an outer rod that are slidably sleeved, the upper surface of the top cover close to the edge is circumferentially provided with a plurality of bumps at equal intervals, the top surface of the bump is arc-shaped, the inner rod corresponds to the bump after extending to lift the telescopic gear lever, one side of the upper surface of the top cover is provided with an opening for delivering hair samples and ultrapure water, and the bottom of the outer side wall of the treatment barrel is provided with a drainage outlet.

As an aspect of the present disclosure, a total of 6 groups of hair cutting assemblies are provided, the tail ends of the auxiliary cutting quartz knife and the cutting quartz knife are aligned, the surface of the cutting quartz knife is provided with a plurality of permeable pores, the surface of the auxiliary cutting quartz knife is provided with a slot, a total of 6 bumps are provided, the auxiliary cutting quartz knife is connected with a clamp groove formed on the outer wall of the rotating outer cylinder through a fixation block, the top and bottom of the fixation block are respectively connected with the top surface and bottom surface of the clamp groove through a first spring rod, the two sides of the outer wall of the rotating inner cylinder are provided with limit strips, the limit strips are both slidably connected with first limit grooves arranged at the two sides of the inside of the rotating outer cylinder and second limit grooves arranged at the two sides of the inner wall of the sleeve, the bottom of the rotating inner cylinder is provided with a weighting block, and the bottom of the weighting block is connected with the inner bottom of the rotating outer cylinder through a second spring rod.

Note: by adjusting the number of the hair cutting assemblies, the maximum cutting efficiency is achieved and the efficiency of washing the hair is not affected, water flow is ensured to pass by arranging the permeable pores and slots so as to strengthen the washing of the hair sample, the arrangement of the first spring rod can play a role in damping the auxiliary quartz knife to a certain extent while providing a certain activity space for the auxiliary quartz knife, the falling back speed of the hair cutting assembly becomes faster by arranging the weighting block so as to improve the cutting efficiency of the hair sample and meanwhile the weighting block can also help completing the press of the auxiliary quartz knife so that the grinding portion of the auxiliary quartz knife located at the bottom of the outer wall of the rotating outer cylinder completes the abutting against the grinding plate.

As an aspect of the present disclosure, the two sides of the upper surface of the pedestal are symmetrically provided with struts, the top of the strut is fixedly connected with the rotating motor through one connection rod, the junction between the connection rod and the rotating motor is provided with a damping spacer, the telescopic rods are arranged below the two connection rods, the bottoms of the two telescopic rods are provided with a limit plate of the inner rod for downward pressing the telescopic gear lever, the tail end of the outer rod is provided with a limit hole, the tail end of the inner rod is provided with a limit protrusion abutted against the limit hole, 3 insulation warehouses are arranged inside the pedestal, temperature adjusters are arranged inside the insulation warehouses, and the two sides of the upper surface of the pedestal are each provided with an observation beaker.

Note: the vibration of the rotating motor can be effectively reduced by arranging the damping spacer, so as to reduce the interference to the hair sample treatment process, the telescopic gear lever can be further pressed through combined arrangement of the telescopic rod and the limit plate so that the cutting quartz knife further presses the auxiliary quartz knife and the grinding portion of the auxiliary quartz knife located at the bottom of the outer wall of the rotating outer cylinder completes the abutting against the grinding plate, the inner rod can be clamped inside the outer rod after shrinking by arranging the limit block and the limit protrusion, so as to avoid the ejection of the inner rod to touch the bump, thereby affecting the grinding.

The present disclosure further provides application of the above method for synchronous extraction and analysis of multiple trace emerging organic contaminants in hairs. The method is applied to a study on long-term exposure of multiple organic contaminants in a human body, and the organic contaminants are PBDEs, OPFRs, PFASs, PAEs and mPAEs.

The present disclosure has the beneficial effects:
(1) The method of the present disclosure optimizes pretreatment conditions of a hair sample, the contaminants in hairs are completely released by grinding treatment, an extraction solution extracting system is established, 5 emerging organic contaminants can be effectively purified and analyzed by establishing a dispersion solid phase extraction filler consisting of Na₂SO₄ powders and C18 powders. The results show that the recovery rates of a total 48 EOCs including 12 PFASs, 11 OPFRs, 9 PAEs, 8 mPAEs and 8 PBDEs are between 53.6% and 138%. The method of the present disclosure only needs 0.1 g of sample and a small amount of organic solvent to obtain good recovery rate and precision, is high in sensitivity, simple and quick, can simultaneously and effectively detect multiple trace emerging organic contaminants in hairs, and provides technical reference for a study on long-term exposure of multiple organic contaminants in a human body.
(2) The device of the present disclosure is based on the method of the present disclosure, can achieve the integration of washing, cutting and grinding on the hair sample, thereby greatly improving the extraction efficiency and saving the workload of the staffs while saving time. The alternate engaged cutting of the cutting quartz knife and the auxiliary quartz knife is completed while rotary washing, and meanwhile the auxiliary quartz knife can be further downward pressed so that the grinding portion of the auxiliary quartz knife located at the bottom of the outer wall of the rotating outer cylinder completes the abutting against the grinding plate.
(3) The application of the present disclosure is based on the method of the present disclosure, treating the hair sample by using the method of the present disclosure can correctly reflect the exposure levels of 5 types of EOCs, and the method of the present disclosure can be applied to analysis on contaminants such as PBDEs, OPFRs, PFASs, PAEs and mPAEs in hairs.

### BRIEF DESCRIPTION OF THE DRAWINGS

FIG. 1 is a diagram of an entire structure of a device according to the present disclosure;

FIG. 2 is a front view and an internal structural diagram of a device according to the present disclosure;

FIG. 3 is a structural diagram of a cutting assembly of a device according to the present disclosure;

FIG. 4 is a diagram of a connection structure between an auxiliary quartz knife and a rotating outer cylinder of a device according to the present disclosure;

FIG. 5 is a diagram of an internal structure of a rotating outer cylinder of a device according to the present disclosure;

FIG. 6 is a sectional view of a sleeve of a device according to the present disclosure;

FIG. 7 is a sectional view of a rotating outer cylinder of a device according to the present disclosure;

FIG. 8 shows recovery rates of PFASs in experimental example in 4 purifying fillers according to the present disclosure;

FIG. 9 shows blank and spike matrix recovery rates of 5 types of compounds measured after being treated in example 1 according to the present disclosure.

Wherein, 1—treatment barrel, 11—grinding plate, 12—top cover, 13—bump, 14—opening, 15—drainage outlet, 2—pedestal, 21—strut, 22—connection rod, 23—insulation warehouse, 24—temperature adjuster, 25—observation beaker, 3—drive assembly, 31—rotating motor, 32—sleeve, 33—second limit groove, 34—damping gasket, 35—telescopic rod, 36—limit plate, 4—rotating outer cylinder, 41—sliding groove, 42—grinding portion, 43—fixation block, 44—clamp groove, 45—first spring rod, 46—first limit groove, 5—rotating inner cylinder, 51—connection block, 52—telescopic gear lever, 53—inner rod, 54—outer rod, 55—limit strip, 56—weighting block, 57—second spring rod, 58—limit hole, 59—limit protrusion, 6—cutting assembly, 61—auxiliary quartz knife, 62—cutting quartz knife, 63—blade, 64—permeable pore, and 65—slot.

### DETAILED DESCRIPTION

### Example 1

A method for synchronous extraction and analysis of multiple trace emerging organic contaminants in hairs comprises the following steps:
S1, pretreatment: a hair sample to be treated together with ultrapure water was added into a treatment barrel 1 in a weight ratio of 1:30, the hair sample was cut through a cutting quartz knife 62 in the treatment barrel 1 while washing for 0.5 h, subsequently ultrapure water was discharged and ultrapure whose weight was 30 times of the weight of the hair sample was added again, the hair sample was cut through the cutting quartz knife 62 in the treatment barrel 1 while washing, until the length of the hair sample was 0.8±0.05 cm, subsequently the ultrapure water was discharged, and then the hair sample was ground for 7.5 min through a grinding plate 11 in the treatment barrel 1 to obtain hair powders;
S2, mixing and extraction: the hair powders obtained in step S1 were dried, 0.1 g of dried hair powders were placed into a centrifuge tube, 1 mL of ethyl acetate and 1 mL of internal standard indicator were added into the centrifuge tube, standing was performed for 12 h, subsequently 4 mL of extract solution was added into the centrifuge tube, wherein the extract solution was prepared from n-hexane, acetone, acetonitrile and ethyl acetate in a volume ratio of 1:1:1:1, centrifugal extraction was performed, wherein the steps for centrifugal extraction were as follows: vortex treatment was carried out for 4 min at the rotation speed of 2000 r·min⁻¹, then ultrasonic treatment was carried out for 20 min at the temperature of 20° C., and finally centrifugal treatment was carried out for 15 min at the rotation speed of 4000 r·min⁻¹, centrifugal extraction was repeated three times, and then the obtained supernatant was merged as a supernatant raw solution, wherein n-hexane, acetone, acetonitrile and ethyl acetate were all standard solutions with concentrations being larger than 99.99%;
a method for preparing the internal standard indicator was as follows: 0.1 mL of 50 g/mL first internal standard raw solution, 0.1 mL of 50 μg/mL second internal standard raw solution, 0.05 mL of 100 μg/mL third internal standard raw solution, 0.05 mL of 100 μg/mL fourth internal standard raw solution and 0.05 mL of 100 μg/mL fifth internal standard raw solution were mixed and then 0.65 mL of methanol solution was added to obtain 1 mL of 5 g/mL mixed internal standard, wherein the first internal standard raw solution was formed by mixing MPFOS and MPFOA in a volume ratio of 1:1, the second internal raw solution was formed by mixing d₁₈-TCIPP, d₁₅-TDCIPP, d₁₅-TPhP and d₁₂-TCEP in a volume ratio of 1:1:1:1, the third internal standard raw solution was formed by mixing d₄-DEHP with d₄-DNBP in a volume ratio of 1:1, the fourth internal standard raw solution was formed by mixing ¹³C₄-mmBP, ¹³C₄-OH-mEHP, ¹³C-mEP, ¹³C-mBzP, d₄-mmOP, 50xo-MEHP-¹³C and MEHP-¹³C₄ in a volume ratio of 1:1:1:1:1:1:1, and the fifth internal standard raw solution was formed by mixing BDE118, BDE128 and ¹³C₁₂-BDE209 in a volume ratio of 1:1:1, and then the mixed internal standard was diluted with a methanol solution to obtain an 100 ng/mL internal standard indicator;
S3, nitrogen blowing concentration and purification: nitrogen blowing concentration was performed on the supernatant raw solution obtained in step S2 to 1 mL, subsequently 20 mg of Na₂SO₄ powders and 100 mg of C18 powders serving as purifying fillers were added, then vortex treatment was carried out for 7 min at the rotation speed of 1200 r·min⁻¹, then centrifugal treatment was carried out for 15 min at the rotation speed of 3500 r·min⁻¹ to obtain a concentrated solution, and then the concentrated solution was transferred to a glass tube; and
S4, low-temperature separation: nitrogen blowing was performed on the concentrated solution obtained in step S3 for 1.5 h, then 200 μL of methanol solution with a mass fraction of ≥99.8% was added for redissolving, standing was performed for 5 h at the temperature of −20° C., the redissolved solution was frozen to remove a precipitate to obtain the hair raw solution after low-temperature separation and LC-MS/MS and GC-MS/MS detection were performed on the hair raw solution.

The treatment barrel 1 for use in this example 1 uses a commercially available corrosion-resistant titanium alloy treatment barrel, the cutting quartz knife 62 uses a commercially available quartz knife with a particle size of 80 #, and the grinding plate 11 uses a commercially available diamond grinding plate.

### Example 2

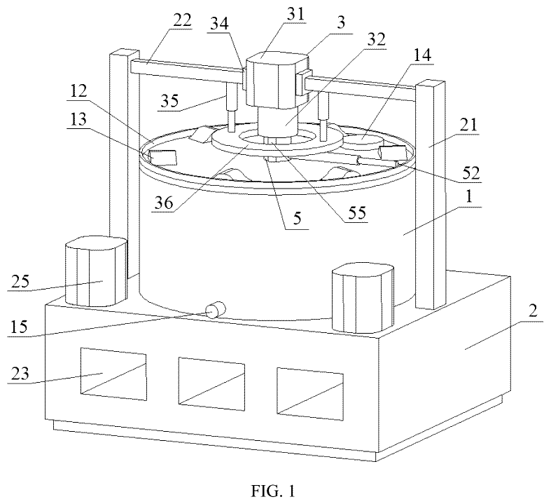

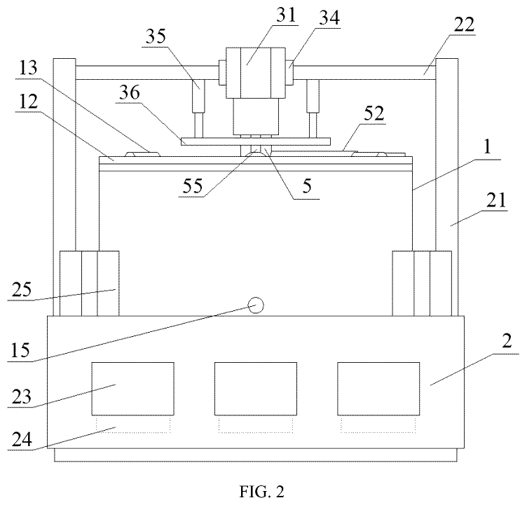

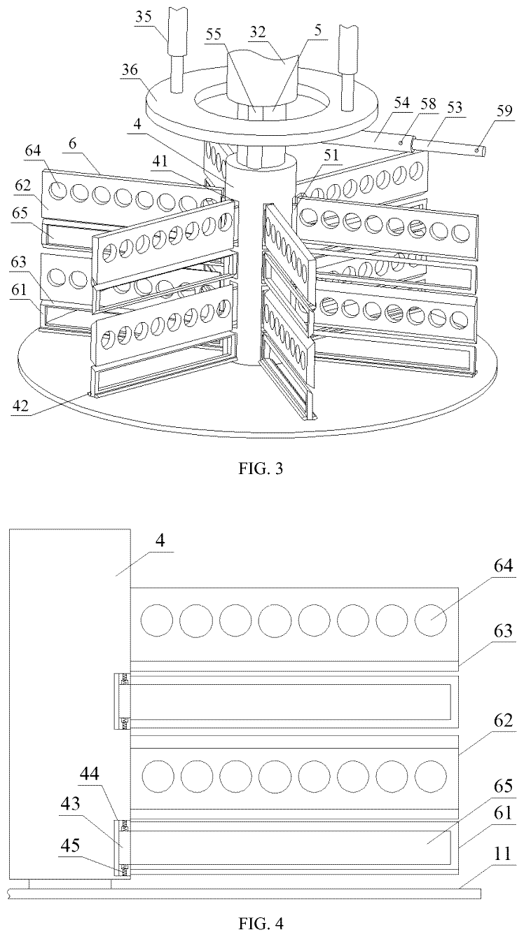

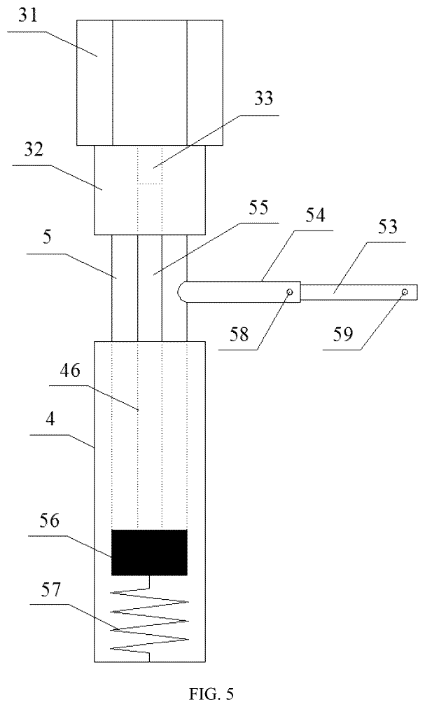

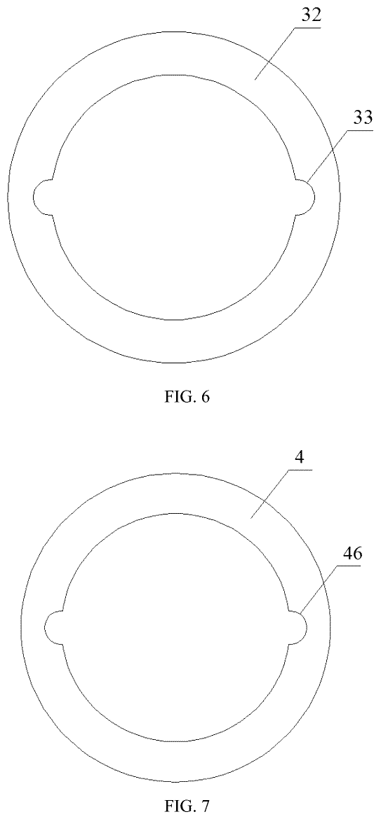

In this example, a device for synchronous extraction and analysis of multiple trace emerging organic contaminants in hairs is provided on the basis of the method in example 1, as shown in FIG. 1, including a treatment barrel 1 for washing, cutting and grinding a hair sample, a pedestal 2 for supporting the treatment barrel 1, and a drive assembly 3 located above the treatment barrel 1;
as shown in FIGS. 1 and 3, the middle inside the treatment barrel 1 is provided with a rotating outer cylinder 4, the inside of the rotating outer cylinder 4 is slidaly sleeved with a rotating inner cylinder 5 that synchronously rotates with the rotating outer cylinder 4, the external circumference of the rotating outer cylinder 4 is provided with a plurality of groups of hair cutting assemblies 6 at equal intervals, the hair cutting assembly 6 includes two auxiliary quartz knives 61 and two cutting quartz knives 62, the auxiliary quartz knives 61 and the cutting quartz knives 62 are alternately arranged, the auxiliary quartz knives 61 are arranged at the bottom and middle of the outer wall of the rotating outer cylinder 4, the upper and lower sides of the auxiliary quartz knives 61 arranged in the middle of the outer wall of the rotating outer cylinder 4 are both provided with sliding grooves 41, a connection block 51 is arranged at the position of the sliding groove 41 corresponding to the outer wall of the rotating inner cylinder 5, the connection block 51 up and down slides along the sliding groove 41, the outer end of the connection block 51 is fixedly connected with the cutting quartz knives 62, the auxiliary quartz knives 61 and the cutting quartz knives 62 both extend to the inner wall close to the treatment barrel 1, the auxiliary quartz knives 61 and the cutting quartz knives 62 on the same group of cutting assemblies 6 are seamlessly engaged and aligned with each other through the blades 63, the blades 63 provided on the cutting quartz knives 62 are alternately engaged and aligned with the blades 63 provided correspondingly on the auxiliary quartz knives 61 when sliding up and down, the bottom of the treatment barrel 1 is provided with a grinding plate 11, and the two sides of the bottoms of the auxiliary quartz knives 61 located at the bottom of the outer wall of the rotating outer cylinder 4 are provided with grinding portions 42 that are matched with the grinding plate 11 to grind the hair samples;
as shown in FIGS. 1, 3 and 5, the top of the treatment barrel 1 is provided with a top cover 12, the top of the rotating inner cylinder 5 penetrates through the top cover 12 and then is connected with the output end of a rotating motor 31 of the drive assembly 3, the output end of the rotating motor 31 is provided with a sleeve 32 that is in up and down sliding connection with the rotating inner cylinder 5, the outer wall of the rotating inner cylinder 5 located above the top cover 12 is provided with a cylindrical telescopic gear lever 52, the telescopic gear lever 52 includes an inner rod 53 and an outer rod 54 that are slidably sleeved, the upper surface of the top cover 12 close to the edge is circumferentially provided with a plurality of bumps at equal intervals, the top surface of the bump 13 is arc-shaped, the inner rod 53 corresponds to the bump 13 after extending to lift the telescopic gear lever 52, one side of the upper surface of the top cover 12 is provided with an opening 14 for delivering hair samples and ultrapure water, and the bottom of the outer side wall of the treatment barrel 1 is provided with a drainage outlet 15, and the rotating motor 31 is a commercially available gear motor;
as shown in FIGS. 4, 5, 6 and 7, the tail ends of the auxiliary cutting quartz knife 61 and the cutting quartz knife 62 are aligned, the surface of the cutting quartz knife 62 is provided with a plurality of permeable pores 64, the surface of the auxiliary cutting quartz knife 61 is provided with a slot 65, a total of 6 bumps 13 are provided, the auxiliary cutting quartz knife 61 is connected with a clamp groove 44 arranged on the outer wall of the rotating outer cylinder 4 through a fixation block 43, the top and bottom of the fixation block 43 are respectively connected with the top surface and bottom surface of the clamp groove 44 through a first spring rod 45, the two sides of the outer wall of the rotating inner cylinder 5 are provided with limit strips 55, the limit strips 55 are both slidably connected with first limit grooves 46 formed at the two sides of the inside of the rotating outer cylinder 4 and second limit grooves 33 formed at the two sides of the inner wall of the sleeve 32, the bottom of the rotating inner cylinder 5 is provided with a weighting block 56, and the bottom of the weighting block 56 is connected with the inner bottom of the rotating outer cylinder 4 through a second spring rod 57;
as shown in FIGS. 1-3, the two sides of the upper surface of the pedestal 2 are symmetrically provided with struts 21, the top of the strut 21 is fixedly connected with the rotating motor 31 through one connection rod 22, the junction between the connection rod 22 and the rotating motor 31 is provided with a damping spacer 34, the telescopic rods 35 are arranged below the two connection rods 22, the bottoms of two telescopic rods 35 are provided with a limit plate 36 of the inner rod 53 for downward pressing the telescopic gear lever 52, the tail end of the outer rod 54 is provided with a limit hole 58, the tail end of the inner rod 53 is provided with a limit protrusion 59 abutted against the limit hole 58, 3 insulation warehouses 23 are arranged inside the pedestal 2, temperature adjusters 24 are arranged inside the insulation warehouses 23, the temperature adjusters 24 are commercially available heating plates, and the two sides of the upper surface of the pedestal 2 are each provided with an observation beaker 25.

The working principle: the working principle of the device of the present disclosure will be simply illustrated.

When in use, the hair sample together with ultrapure water is added to the treatment barrel 1 through the opening 14. The rotating motor 31 is turned on to drive the sleeve 32, the rotating inner cylinder 5 and the rotating outer cylinder 4 simultaneously rotate. At the same time, during the rotation, the inner rod 53 of the telescopic gear lever 52 extends and intermittently slides over each bump 13 so that the telescopic gear lever intermittently rises and falls, thereby driving the rotating inner cylinder 5 so that the connection block 51 intermittently rises and falls in the sliding groove 41, and the blades 63 provided on the shear quartz knives 62 are alternately engaged and aligned with the blades 63 correspondingly provided on the auxiliary quartz knives 61 when the cutting quartz knives 62 slide up and down to complete the cutting and crushing of the hair sample;
the arrangement of permeable pores 64 and slots 65 ensures water flow to pass while cutting and crushing, thereby strengthening the cleaning of the hair sample. After cleaning is ended, the drainage hole 15 is opened to drain up the water and prepare for the grinding of the hair sample;
the inner rod 53 is shrunk so that the limit protrusion 59 is abutted against with the limit hole 58, and the inner rod 53 retracts into the outer rod 54 and engaged with the outer rod 54. At this time, the telescopic gear lever 52 no longer touches the bump 13 when rotating, and at the same time, the telescopic rod 35 declines, and the limit plate 36 downward presses the telescopic gear lever 52, so that the fixation block 43 is further descended inside the clamp groove 44, the first spring rod 45 is downward pressed, and the weighting block 56 downward presses the second spring rod 57 so that the grinding portion 42 further fits with the grinding plate 11, and then the rotating motor 31 is turned on to achieve the grinding of the hair sample.

### Example 3

In this example, application of the method for synchronous extraction and analysis of multiple trace emerging organic contaminants in hairs is provided on the basis of the method in example 1. The method for synchronous extraction and analysis of multiple trace emerging organic contaminants in hairs is applied to a study on long-term exposure of multiple organic contaminants in a human body, and the organic contaminants are PBDEs, OPFRs, PFASs, PAEs and mPAEs.

### Example 4

This example is different from example 1 that method parameters for pretreatment in step S1 are different.

S1, pretreatment: a hair sample to be treated together with ultrapure water was added into a treatment barrel 1 in a weight ratio of 1:20, the hair sample was cut through a cutting quartz knife 62 in the treatment barrel 1 while washing for 0.5 h, subsequently ultrapure water was discharged and ultrapure whose weight was 20 times of the weight of the hair sample was added again, the hair sample was cut through the cutting quartz knife 62 in the treatment barrel 1 while washing, until the length of the hair sample was 0.55±0.05 cm, subsequently the ultrapure water was discharged, and then the hair sample was ground for 5 min through a grinding plate 11 in the treatment barrel 1 to obtain hair powders.

### Example 5

This example is different from example 1 that method parameters for pretreatment in step S1 are different.

S1, pretreatment: a hair sample to be treated together with ultrapure water was added into a treatment barrel 1 in a weight ratio of 1:40, the hair sample was cut through a cutting quartz knife 62 in the treatment barrel 1 while washing for 1 h, subsequently ultrapure water was discharged and ultrapure whose weight was 40 times of the weight of the hair sample was added again, the hair sample was cut through the cutting quartz knife 62 in the treatment barrel 1 while washing, until the length of the hair sample was 0.95±0.05 cm, subsequently the ultrapure water was discharged, and then the hair sample was ground for 10 min through a grinding plate 11 in the treatment barrel 1 to obtain hair powders.

### Example 6

This example is different from example 1 that method parameters for low-temperature separation in step S4 are different.

S4, low-temperature separation: nitrogen blowing was performed on the concentrated solution obtained in step S3 for 1 h, then 200 μL of methanol solution with a mass fraction of ≥99.9% was added for redissolving, standing was performed for 4 h at the temperature of −20° C., the redissolved solution was frozen, a precipitate was removed to obtain the hair raw solution after low-temperature separation, and LC-MS/MS and GC-MS/MS detection were performed on the hair raw solution.

### Example 7

This example is different from example 1 that method parameters for low-temperature separation in step S4 are different.

S4, low-temperature separation: nitrogen blowing was performed on the concentrated solution obtained in step S3 for 2 h, then 200 μL of methanol solution with a mass fraction of ≥99.9% was added for redissolving, standing was performed for 6 h at the temperature of −20° C., the redissolved solution was frozen, a precipitate was removed to obtain the hair raw solution after low-temperature separation, and LC-MS/MS and GC-MS/MS detection were performed on the hair raw solution.

### EXPERIMENTAL EXAMPLE

The authenticity and effectiveness of the method of the present disclosure, comparative tests were conducted and three groups of comparative examples were selected for comparative analysis. The methods in 3 groups of comparative examples were completely the same as the method in example 1, except for the selection of extraction solutions and purifying fillers, wherein, in comparative example 1, the extraction solutions are ACE, HEX and MTBE, and the purifying fillers are anhydrous MgSO₄, PSA and C18; in comparative example 2, the extraction solutions are ACE, HEX and MTBE, and the purifying fillers are anhydrous Na₂SO₄ and C18; in comparative example 3, the extraction solutions are EtAC, HEX and MTBE, and the purifying fillers were anhydrous Na₂SO₄ and C18.

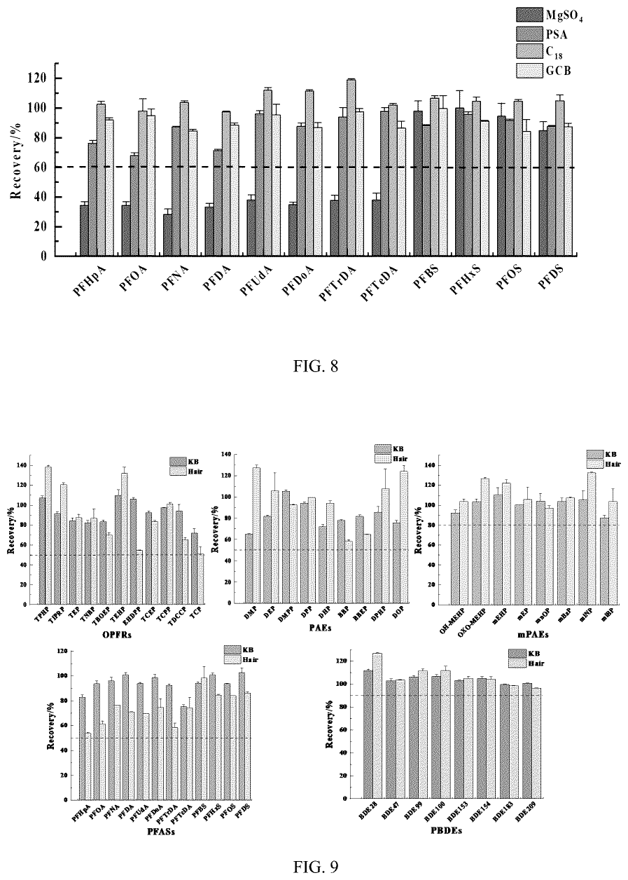

First, to verify the effect of the selected purifying filler of the present disclosure on PFASs, MgSO₄, PSA, C18 and GCB were selected for purification after solvent addition. As shown in FIG. 8, the overall recovery rate of PFASs showed a trend of C18>GCB>PSA>MgSO₄. Wherein, after being treated with MgSO₄, the recovery rates of 4 perfluoroalkyl acids are all below 50%, so it is speculated that MgSO₄ may have a strong adsorption effect on PFAS contaminants, so the recovery rates are relatively poor. MgSO₄, as a desiccant, can absorb trace amounts of water in the solution. Considering the possible adsorption effect, Na₂SO₄ is used in subsequent experiments. C18 is generally used to remove non-polar impurities such as fats, and research results show that it has the least impact on most compounds. Therefore, the present disclosure preferably selects Na₂SO₄ and C18 as purifying fillers.

After the purifying filler is determined, the selection of extraction solution is further optimized. Alves et al. compared the extraction efficiencies of PFASs on ethyl acetate, isopropanol, and tetrahydrofuran/isopropanol (50:50, V/V), and found that ethyl acetate has the best effect (recovery rate between 69% and 141%). In addition, ethyl acetate is often used as an excellent extractant or eluent for OPFRs, which can achieve good recovery results. Therefore, the present disclosure compares the extraction efficiencies of 5 types of contaminants in comparative example 2 and comparative example 3, respectively. After treatment in comparative example 2, EHDPP, PFHpA, and PFOA have relatively poor recovery rates ranging from 29.6% to 42.8%; however, after treatment in comparative example 2, the recovery rates of EHDPP and PFBS are between 21.42% and 48.22%, and such the two solutions have certain defects.

Based on the above experimental results, considering that mPAEs are hydrophilic compounds, and therefore the extraction solution proportion in example 1 is further used, and Na₂SO₄ and C18 are used as purifying fillers for hair treatment. The results are shown in FIG. 9. The spiked recovery rates of PBDEs, PFASs, OPFRs, PAEs and mPAEs matrices range from 53.6% to 138%, with RSD: 0.01-18.9%. The overall recovery rate of the compound is significantly improved than that before. It is noted that PFDA, EHDPP, TCP and other compounds with the recovery rates of PFASs and OPFRs being always lower than 40% have significantly improved effects after being treated with the method in example 1, and the recovery rates and the relative standard deviations are 53.6-138% and 0.01-9.29%, respectively.

1. A method for synchronous extraction and analysis of multiple trace emerging organic contaminants in hairs, comprising the following steps:
S1, pretreatment: adding a hair sample to be treated together with ultrapure water into a treatment barrel (1) in a weight ratio of 1:20-40, cutting the hair sample through a cutting quartz knife (62) in the treatment barrel (1) while washing for 0.5-1 h, subsequently discharging ultrapure water and adding ultrapure whose weight is 20-40 times of the weight of the hair sample again, cutting the hair sample through the cutting quartz knife (62) in the treatment barrel (1) while washing, until the length of the hair sample is 0.5-1 cm, subsequently discharging the ultrapure water, and then grinding the hair sample for 5-10 min through a grinding plate (11) in the treatment barrel (1) to obtain hair powders;
S2, mixing and extraction: drying the hair powders obtained in step S1, placing 0.1 g of dried hair powders into a centrifuge tube, adding 1 mL of ethyl acetate and 1 mL of internal standard indicator into the centrifuge tube, standing for 12 h, and then adding 4 mL of extract solution into the centrifuge tube, wherein the extract solution is prepared from n-hexane, acetone, acetonitrile and ethyl acetate in a volume ratio of 1:1:1:1, and then starting to perform centrifugal extraction, wherein the steps for centrifugal extraction are as follows: vortex treatment is carried out for 4 min at the rotation speed of 2000 r·min⁻¹, then ultrasonic treatment is carried out for 20 min at the temperature of 20° C., and finally centrifugal treatment is carried out for 15 min at the rotation speed of 4000 r·min⁻¹, centrifugal extraction is repeated 3 times, and then the obtained supernatant is merged as a supernatant raw solution;
S3, nitrogen blowing concentration and purification: performing nitrogen blowing concentration on the supernatant raw solution obtained in step S2 to 1 mL, subsequently adding 20 mg of Na₂SO₄ powders and 100 mg of C18 powders serving as purifying fillers, then performing vortex treatment for 7 min at the rotation speed of 1200 r·min⁻¹, then performing centrifugal treatment for 15 min at the rotation speed of 3500 r·min⁻¹ to obtain a concentrated solution, and then transferring the concentrated solution to a glass tube; and
S4, low-temperature separation: blowing the concentrated solution obtained in step S3 with nitrogen for 1-2 h, then adding 200 μL of methanol solution with a mass fraction of ≥99.8% for redissolving, standing for 4-6 h at the temperature of −20° C., freezing and removing a precipitate to obtain the hair raw solution after low-temperature separation and performing Liquid chromatography-tandem mass spectrometry/mass spectrometry (LC-MS/MS) and Gas chromatography-tandem mass spectrometry/mass spectrometry (GC-MS/MS) detection analysis on the hair raw solution.

2. The method according to claim 1, wherein a method for preparing the internal standard indicator in the step S2 is as follows: mixing 0.1 mL of 50 μg/mL first internal standard raw solution, 0.1 mL of 50 μg/mL second internal standard raw solution, 0.05 mL of 100 μg/mL third internal standard raw solution, 0.05 mL of 100 μg/mL fourth internal standard raw solution and 0.05 mL of 100 μg/mL fifth internal standard raw solution and then adding 0.65 mL of methanol solution to obtain 1 mL of 5 μg/mL mixed internal standard, wherein the first internal standard raw solution is formed by mixing MPFOS and MPFOA in a volume ratio of 1:1, the second internal raw solution is formed by mixing d₁₈-TCIPP, d₁₅-TDCIPP, d₁₅-TPhP and d₁₂-TCEP in a volume ratio of 1:1:1:1, the third internal standard raw solution is formed by mixing d₄-DEHP with d₄-DNBP in a volume ratio of 1:1, the fourth internal standard raw solution is formed by mixing ¹³C₄-mmBP, ¹³C₄—OH-mEHP, ¹³C-mEP, ¹³C-mBzP, d₄-mmOP, 50xo-MEHP-¹³C and MEHP-¹³C₄ in a volume ratio of 1:1:1:1:1:1:1, and the fifth internal standard raw solution is formed by mixing BDE118, BDE128 and ¹³C₁₂-BDE209 in a volume ratio of 1:1:1; and then diluting the mixed internal standard with a methanol solution to obtain an 100 ng/mL internal standard indicator.

3. A device for use in the method according to claim 2, comprising a treatment barrel (1) for washing, cutting and grinding a hair sample, a pedestal (2) for supporting the treatment barrel (1), and a drive assembly (3) located above the treatment barrel (1);
wherein, the middle inside the treatment barrel (1) is provided with a rotating outer cylinder (4), the inside of the rotating outer cylinder (4) is slidaly sleeved with a rotating inner cylinder (5) that synchronously rotates with the rotating outer cylinder (4), the external circumference of the rotating outer cylinder (4) is provided with a plurality of groups of hair cutting assemblies (6) at equal intervals, the hair cutting assembly (6) includes two auxiliary quartz knives (61) and two cutting quartz knives (62), the auxiliary quartz knives (61) and the cutting quartz knives (62) are alternately arranged, the auxiliary quartz knives (61) are arranged at the bottom and middle of the outer wall of the rotating outer cylinder (4), the upper and lower sides of the auxiliary quartz knives (61) arranged in the middle of the outer wall of the rotating outer cylinder (4) are both provided with sliding grooves (41), a connection block (51) is arranged at the position of the sliding groove (41) corresponding to the outer wall of the rotating inner cylinder (5), the connection block (51) up and down slides along the sliding groove (41), the outer end of the connection block (51) is fixedly connected with the cutting quartz knives (62), the auxiliary quartz knives (61) and the cutting quartz knives (62) both extend to the inner wall close to the treatment barrel (1), the auxiliary quartz knives (61) and the cutting quartz knives (62) on the same group of cutting assemblies (6) are seamlessly engaged and aligned with each other through the blades (63), the blades (63) provided on the cutting quartz knives (62) are alternately engaged and aligned with the blades (63) provided correspondingly on the auxiliary quartz knives (61) when sliding up and down, the bottom of the treatment barrel (1) is provided with a grinding plate (11), and the two sides of the bottoms of the auxiliary quartz knives (61) located at the bottom of the outer wall of the rotating outer cylinder (4) are provided with grinding portions (42) that are matched with the grinding plate (11) to grind the hair samples;
the top of the treatment barrel (1) is provided with a top cover (12), the top of the rotating inner cylinder (5) penetrates through the top cover (12) and then is connected with the output end of a rotating motor (31) of the drive assembly (3), the output end of the rotating motor (31) is provided with a sleeve (32) that is in up and down sliding connection with the rotating inner cylinder (5), the outer wall of the rotating inner cylinder (5) located above the top cover (12) is provided with a cylindrical telescopic gear lever (52), the telescopic gear lever (52) includes an inner rod (53) and an outer rod (54) that are slidably sleeved, the upper surface of the top cover (12) close to the edge is circumferentially provided with a plurality of bumps (13) at equal intervals, the top surface of the bump (13) is arc-shaped, the inner rod (53) corresponds to the bump (13) after extending to lift the telescopic gear lever (52), one side of the upper surface of the top cover (12) is provided with an opening (14) for delivering hair samples and ultrapure water, and the bottom of the outer side wall of the treatment barrel (1) is provided with a drainage outlet (15).

4. The device according to claim 3, wherein a total of 6 groups of hair cutting assemblies (6) are provided, the tail ends of the auxiliary cutting quartz knife (61) and the cutting quartz knife (62) are aligned, the surface of the cutting quartz knife (62) is provided with a plurality of permeable pores (64), the surface of the auxiliary cutting quartz knife (61) is provided with a slot (65), a total of 6 bumps are provided, the auxiliary cutting quartz knife (61) is connected with a clamp groove (44) arranged on the outer wall of the rotating outer cylinder (4) through a fixation block (43), the top and bottom of the fixation block (43) are respectively connected with the top surface and bottom surface of the clamp groove (44) through a first spring rod (45), the two sides of the outer wall of the rotating inner cylinder (5) are provided with limit strips (55), the limit strips (55) are both slidably connected with first limit grooves (46) arranged at the two sides of the inside of the rotating outer cylinder (4) and second limit grooves (33) arranged at the two sides of the inner wall of the sleeve (32), the bottom of the rotating inner cylinder (5) is provided with a weighting block (56), the bottom of the weighting block (56) is connected with the inner bottom of the rotating outer cylinder (4) through a second spring rod (57).

5. The device according to claim 3, wherein the two sides of the upper surface of the pedestal (2) are symmetrically provided with struts (21), the top of the strut (21) is fixedly connected with the rotating motor (31) through one connection rod (22), the junction between the connection rod (22) and the rotating motor (31) is provided with a damping spacer (34), the telescopic rods (35) are arranged below the two connection rods (22), the bottoms of the two telescopic rods (35) are provided with a limit plate (36) of the inner rod (53) for downward pressing the telescopic gear lever (52), the tail end of the outer rod (54) is provided with a limit hole (58), the tail end of the inner rod (53) is provided with a limit protrusion (59) abutted against the limit hole (58), 3 insulation warehouses (23) are arranged inside the pedestal (2), temperature adjusters (24) are arranged inside the insulation warehouses (23), and the two sides of the upper surface of the pedestal (2) are each provided with an observation beaker (25).

6. A device for use in the method according to claim 1, comprising a treatment barrel (1) for washing, cutting and grinding a hair sample, a pedestal (2) for supporting the treatment barrel (1), and a drive assembly (3) located above the treatment barrel (1);
wherein, the middle inside the treatment barrel (1) is provided with a rotating outer cylinder (4), the inside of the rotating outer cylinder (4) is slidaly sleeved with a rotating inner cylinder (5) that synchronously rotates with the rotating outer cylinder (4), the external circumference of the rotating outer cylinder (4) is provided with a plurality of groups of hair cutting assemblies (6) at equal intervals, the hair cutting assembly (6) includes two auxiliary quartz knives (61) and two cutting quartz knives (62), the auxiliary quartz knives (61) and the cutting quartz knives (62) are alternately arranged, the auxiliary quartz knives (61) are arranged at the bottom and middle of the outer wall of the rotating outer cylinder (4), the upper and lower sides of the auxiliary quartz knives (61) arranged in the middle of the outer wall of the rotating outer cylinder (4) are both provided with sliding grooves (41), a connection block (51) is arranged at the position of the sliding groove (41) corresponding to the outer wall of the rotating inner cylinder (5), the connection block (51) up and down slides along the sliding groove (41), the outer end of the connection block (51) is fixedly connected with the cutting quartz knives (62), the auxiliary quartz knives (61) and the cutting quartz knives (62) both extend to the inner wall close to the treatment barrel (1), the auxiliary quartz knives (61) and the cutting quartz knives (62) on the same group of cutting assemblies (6) are seamlessly engaged and aligned with each other through the blades (63), the blades (63) provided on the cutting quartz knives (62) are alternately engaged and aligned with the blades (63) provided correspondingly on the auxiliary quartz knives (61) when sliding up and down, the bottom of the treatment barrel (1) is provided with a grinding plate (11), and the two sides of the bottoms of the auxiliary quartz knives (61) located at the bottom of the outer wall of the rotating outer cylinder (4) are provided with grinding portions (42) that are matched with the grinding plate (11) to grind the hair samples;
the top of the treatment barrel (1) is provided with a top cover (12), the top of the rotating inner cylinder (5) penetrates through the top cover (12) and then is connected with the output end of a rotating motor (31) of the drive assembly (3), the output end of the rotating motor (31) is provided with a sleeve (32) that is in up and down sliding connection with the rotating inner cylinder (5), the outer wall of the rotating inner cylinder (5) located above the top cover (12) is provided with a cylindrical telescopic gear lever (52), the telescopic gear lever (52) includes an inner rod (53) and an outer rod (54) that are slidably sleeved, the upper surface of the top cover (12) close to the edge is circumferentially provided with a plurality of bumps (13) at equal intervals, the top surface of the bump (13) is arc-shaped, the inner rod (53) corresponds to the bump (13) after extending to lift the telescopic gear lever (52), one side of the upper surface of the top cover (12) is provided with an opening (14) for delivering hair samples and ultrapure water, and the bottom of the outer side wall of the treatment barrel (1) is provided with a drainage outlet (15).

7. The device according to claim 6, wherein a total of 6 groups of hair cutting assemblies (6) are provided, the tail ends of the auxiliary cutting quartz knife (61) and the cutting quartz knife (62) are aligned, the surface of the cutting quartz knife (62) is provided with a plurality of permeable pores (64), the surface of the auxiliary cutting quartz knife (61) is provided with a slot (65), a total of 6 bumps are provided, the auxiliary cutting quartz knife (61) is connected with a clamp groove (44) arranged on the outer wall of the rotating outer cylinder (4) through a fixation block (43), the top and bottom of the fixation block (43) are respectively connected with the top surface and bottom surface of the clamp groove (44) through a first spring rod (45), the two sides of the outer wall of the rotating inner cylinder (5) are provided with limit strips (55), the limit strips (55) are both slidably connected with first limit grooves (46) arranged at the two sides of the inside of the rotating outer cylinder (4) and second limit grooves (33) arranged at the two sides of the inner wall of the sleeve (32), the bottom of the rotating inner cylinder (5) is provided with a weighting block (56), the bottom of the weighting block (56) is connected with the inner bottom of the rotating outer cylinder (4) through a second spring rod (57).

8. The device according to claim 6, wherein the two sides of the upper surface of the pedestal (2) are symmetrically provided with struts (21), the top of the strut (21) is fixedly connected with the rotating motor (31) through one connection rod (22), the junction between the connection rod (22) and the rotating motor (31) is provided with a damping spacer (34), the telescopic rods (35) are arranged below the two connection rods (22), the bottoms of the two telescopic rods (35) are provided with a limit plate (36) of the inner rod (53) for downward pressing the telescopic gear lever (52), the tail end of the outer rod (54) is provided with a limit hole (58), the tail end of the inner rod (53) is provided with a limit protrusion (59) abutted against the limit hole (58), 3 insulation warehouses (23) are arranged inside the pedestal (2), temperature adjusters (24) are arranged inside the insulation warehouses (23), and the two sides of the upper surface of the pedestal (2) are each provided with an observation beaker (25).

9. Application of the method according to claim 1, wherein the method for synchronous extraction and analysis of multiple trace emerging organic contaminants in hairs is applied to a study on long-term exposure of multiple organic contaminants in a human body, and the organic contaminants are PBDEs, OPFRs, PFASs, PAEs and mPAEs.
